(19) United States
(12) Patent Application Publication
(10) Pub. No.: US 2026/0207041 A1
(43) Pub. Date: Jul. 23, 2026

(54) Laryngoscope

(71) Applicant: Verathon Inc., Bothell, WA (US)

(72) Inventor: Mackenzie Spence, Burnaby (CA)

(73) Assignee: Verathon Inc., Bothell, WA (US)

(21) Appl. No.: 19/451,240

(22) Filed: Jan. 16, 2026

Related U.S. Application Data
(60) Provisional application No. 63/746,810, filed on Jan. 17, 2025.

(51) Int. Cl. A61B 1/267; A61B 1/00

(52) U.S. Cl. CPC A61B 1/267; A61B 1/0008

(57) ABSTRACT

Laryngoscopes may include surface finishes having a VDI 3400 Standard grade value of less than about 33 corresponding to an average roughness (RA) of less than about 4.5μm. Surface finishes may be present on one or more portions of a laryngoscope. An upper surface of a blade of a laryngoscope may include a surface finish on at least a portion of the blade having a VDI 3400 standard grade value in a range from about 15 to about 30. An upper surface of a blade may include a surface finish on at least a portion of the blade having a VDI 3400 standard grade value in a range from about 21 to about 24. A blade of a laryngoscope may include a surface finish on at least a portion of the blade having a VDI 3400 standard grade value in a range from about 27 to about 30.

### CROSS-REFERENCE TO RELATED APPLICATIONS

This application claim benefit to U.S. Provisional Ser. No. 63/746,810 filed on Jan. 17, 2025, entitled “Laryngoscope,” which is incorporated herein by reference in its entirety.

### FIELD OF THE INVENTION

This invention relates to a laryngoscope having an improved design.

### BACKGROUND OF THE INVENTION

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Citation or identification of any document in this application is not an admission that such document is available as prior art to the present invention.

During medical procedures it may be necessary or desirable to examine and/or clear the mouth and throat of a patient using a laryngoscope. For example, laryngoscopes are used in endotracheal intubations during surgery.

Generally, laryngoscopes include an elongate blade which is attached to a handle. A laryngoscope blade may be inserted through the mouth of the patient into the pharyngeal area. During insertion portions of the laryngoscope may contact soft tissue of the oral mucosa such as the tongue or cheeks and pharyngeal area such as the epiglottis, epiglottic vallecula, esophagus, vocal cords, and/or the trachea.

Types of blade constructions generally used in laryngoscopes include a straight blade (i.e., “Miller blade,” “Phillips blade,” “Wis-Hipple”) and a curved blade (i.e., “MacIntosh blade” or a hyperangulated blade). As shown in the schematic in FIG. 1, a laryngoscope 2 having a curved MacIntosh blade 4 is positioned in the throat of a patient such that the blade 4 is positioned proximate tongue 6. In contrast, FIG. 2 depicts a schematic of a laryngoscope 8 having a straight Miller blade 10 is positioned in throat of a patient such that the blade 10 engages both the tongue 6 and epiglottis 12. Use of a straight blade may provide a user (i.e., a medical provider) an improved view of vocal chords during intubation. In particular, use of a straight blade as illustrated in FIG. 2 may provide a user an improved view of vocal chords 14 during intubation by moving epiglottis 12 out of the field of vision of the user.

Laryngoscopes usually include a smooth surface designed to allow for ease of entry of the laryngoscope into the mouth and throat. Further, smooth surfaces have typically been used to reduce and/or inhibit damage to the soft tissue that may come in contact with a portion of the laryngoscope. However, use of smooth surfaces on the laryngoscope may allow the laryngoscope to slip relative to the tissues during use. Any slippage during an intubation, for example, can be a nuisance for the physician and detrimental to patient care.

In particular, patients with smoother tissues and/or higher amounts of fluids in mouth may be at an increased risk for slippage a laryngoscope is used. For example, babies may be at an increased risk for slippage during intubation as their tissues are generally smoother than adults. Further due to the size constraints of a baby's mouth and airway, generally, the laryngoscopes used for babies and small children are generally narrower allowing for less surface area contacting the tissues of interest.

In particular, this can be a problem for laryngoscopes having a Miller blade that engages the epiglottis as is shown in FIG. 2. Babies generally have smoother tissues in the areas that contact the blade than older patients and are therefore at additional risk for slippage of the laryngoscope blade during use. For example, trapping the epiglottis with a Miller blade in a child, baby, in particular neonate is complicated by use of smooth blades.

### SUMMARY OF THE INVENTION

Reducing and/or inhibiting slippage during use of a laryngoscope may be accomplished by changes to the surfaces of the laryngoscope. Finishes may be applied, molded into, adhered to, or by using any method or combination of methods known in the art to surfaces of a laryngoscope, or elements thereof, such that slippage is reduced and/or inhibited while maintaining the ease of entry of the blade into the mouth and/or the pharyngeal area of patients.

In an embodiment, the area of the laryngoscope that comes into contact with the patient's epiglottis and/or tongue includes a surface finish that reduces and/or inhibits slippage of the laryngoscope. The surface finish may provide grip, but does not act as an abrasive surface, which may damage tissues of the patient. In some instances, a surface finish of a portion of a blade of a laryngoscope may engage at least a portion of an epiglottis of a patient during use. Alternately, a surface finish of a portion of the upper surface of the blade may engage at least a portion of an epiglottis of a patient during use.

Portions of a laryngoscope may have a predetermined coefficient of friction depending on characteristics of a patient such as age, condition, procedure, and/or preferences of the provider.

Laryngoscopes as described herein may have surfaces having a Verein Deutscher Ingenieure (VDI) 3400 Standard grade value of less than about 33. In some embodiments, one or more surfaces of a laryngoscope may have a Verein Deutscher Ingenieure (VDI) 3400 Standard grade value of less than about 30. In a particular instance, all surfaces of a laryngoscope may have a surface finishes having a VDI 3400 Standard grade value of less than about 30. For example, all surfaces on a blade of a laryngoscope may have a VDI 3400 Standard grade value of less than about 30. In a further example, all surfaces on a blade of a laryngoscope may have a VDI 3400 Standard grade value of less than about 33.

An embodiment may include a laryngoscope with a blade having both an upper surface having a surface finish on at least part of the upper surface and a lower blade surface. In a particular example, an upper blade may have a surface finish having a VDI 3400 Standard grade value in a range from about 15 to about 33. The surface finish on the upper blade surface may have a VDI 3400 Standard grade value in a range from about 15 to about 30. The surface finish portion of the upper blade surface may engage at least a portion of an epiglottis of a patient during use.

An embodiment of a laryngoscope may include a blade that has an angled portion. In some instances, the angled portion may include one or more surface finishes. An upper surface of an angled portion of the blade may include a surface finish that differs from a surface finish on the underside and/or sides of the blade.

An embodiment may include an upper blade surface with a surface finish having a VDI 3400 Standard grade value in a range from about 15 to about 30. In a further embodiment, an upper blade surface may have a surface finish having a VDI 3400 Standard grade value in a range from about 18 to about 30.

In a particular instance, an upper blade surface may have a surface finish portion that includes a surface finish having a VDI 3400 Standard grade value in a range from about 18 to about 27. In a further embodiment, an upper blade surface may have a surface finish portion that includes a surface finish having a VDI 3400 Standard grade value in a range from about 27 to about 30. In a particular example, the surface finish on the upper surface of the blade may have a VDI 3400 Standard grade value in a range from about 27 to about 33. In some embodiments, the surface finish on the upper surface of the blade may have a VDI 3400 Standard grade value in a range from about 21 to about 24.

A lower blade surface may include a surface finish portion having a surface finish having a VDI 3400 Standard grade value in a range from about 15 to about 30. Some embodiments may include a lower blade surface having a surface finish portion that includes a surface finish having a VDI 3400 Standard grade value in a range from about 18 to about 27. In a further embodiment, a lower blade surface may have a surface finish portion that includes a surface finish having a VDI 3400 Standard grade value in a range from about 18 to about 30. In a particular example, a lower blade surface may include a surface finish portion having a surface finish having a VDI 3400 Standard grade value in a range from about 21 to about 24. In a further example, a lower blade surface may include a surface finish portion having a surface finish having a VDI 3400 Standard grade value in a range from about 27 to about 30. In another example, a lower blade surface may include a surface finish portion having a surface finish having a VDI 3400 Standard grade value in a range from about 27 to about 33. Alternately, a lower blade surface may include a surface finish portion having a surface finish having a VDI 3400 Standard grade value in a range from about 18 to about 21.

In some embodiments, portions of a laryngoscope may have predetermined average roughness values for surfaces. Embodiments of a laryngoscope may have surfaces having an average roughness of less than about 4.5 μm. Surfaces of a laryngoscope may have a surface finish having an average roughness (RA) of less than about 3.15 μm. In a particular embodiment, all surfaces of the laryngoscope have an average roughness (RA) of less than about 3.15 μm. For example, in some embodiments, external surfaces of a laryngoscope, that is, surfaces of a laryngoscope that may contact tissues, have an average roughness of less than about 3.15 μm.

Embodiments of a laryngoscope may a blade where all external surfaces have an average roughness of less than about 4.5 μm. In an embodiment, a laryngoscope may have a blade where all external surfaces have an average roughness in a range from about 0.56 μm to about 3.15 μm. In a particular instance, a blade may include all external surfaces having a surface average roughness in a range from about 0.8 μm to about 2.24 μm. For example, in some embodiments a laryngoscope may have a blade having all external surfaces having an average roughness in a range from about 1.12 μm to about 1.6 μm. In a further embodiment, a laryngoscope blade may have an average roughness in a range from about 0.8 μm to about 3.15 μm on all external surfaces of the blade.

For example, a laryngoscope may include a blade having a first portion having a particular value for surface average roughness. Further, a second portion may of the blade may have a second predetermined value of surface average roughness (RA). In some embodiments, the first portion may have a surface average roughness that is greater than a surface average roughness of the second portion.

Laryngoscopes may include portions on a blade having a surface average roughness in a range from about 0.56 μm to about 4.5 μm. Some embodiments of laryngoscopes have one or more portions of a blade that may have a surface average roughness is in a range from about 0.56 μm to about 3.15 μm. In a further embodiment, portions of a blade may have an average surface roughness in a range from about 0.8 to about 3.15 um. In a particular instance, portions of a blade may have a surface average roughness in a range from about 0.8 μm to about 2.24 μm. For example, in some embodiments a laryngoscope may have one or more portions having surface average roughness in a range from about 1.12 μm to about 1.6 μm. In a further example, portions of a blade may have an average surface roughness in a range from about 2.2 to about 3.15 um.

Embodiments of a laryngoscope may have surface finishes on lower surface a blade having an average roughness of less than about 4.5 μm. In some embodiments, portions of laryngoscope, in particular, portions on a lower surface of a blade may have an average roughness of less than about 3.15 μm. In a particular embodiment, a portion on a lower surface of the blade may have an average roughness in a range from about 0.8 μm to about 2.24 μm. For example, a portion of the lower surface of the blade may have an average roughness in a range from about 1.12 μm to about 1.6 μm. In a further example, a portion of the lower surface of the blade may have an average roughness in a range from about 2.2 μm to about 3.15 μm.

In some embodiments, a portion of a handle and a portion blade may be a unitary construction formed during the manufacturing process.

A laryngoscope for insertion into an airway of a patient may include a blade having a portion on an upper surface that has a surface finish. For example, in some instances a whole upper surface of the blade (e.g., including edges and sides) up to a line separating the blade from the handle portion may include a surface finish. A configuration on a surface finish on a blade may be positioned such that the blade could be used for a wide range of patients. Portions of a laryngoscope may have a predetermined coefficient of friction depending on characteristics of a patient such as age, condition, procedure, and/or preferences of the provider.

In some instances, a laryngoscope may have a lower surface of the blade having a predetermined reflectivity based on preferences of users. The laryngoscope includes a handle coupled to the blade.

In some embodiments, surface finishes may have patterning including but not limited to chevrons, polygons, rounded forms, parallel lines, intersecting lines, random spots, etc. and combinations thereof.

An embodiment of a laryngoscope for use in a patient's airway may be formed from polymeric material. The laryngoscope may include a blade having a straight configuration and a handle coupled to the blade. A surface finish on a portion of the blade may have a grade value in a range from about 21 to about 24 when measured according to Verein Deutscher Ingenieure (VDI) 3400 Standard. An upper surface of the blade engages the epiglottis of a patient during use to reduce movement of the epiglottis. In some instances, the upper surface of the blade is used to pin the epiglottis such that a view of the vocal chords and/or trachea is improved for a user during use of the laryngoscope.

A further embodiment of a laryngoscope may include a blade having a straight configuration having a surface finish on a portion of the blade may have a grade value in a range from about 27 to about 30 when measured according to Verein Deutscher Ingenieure (VDI) 3400 Standard.

In a particular embodiment, a laryngoscope may include a distal end positionable proximate the throat region of a patient during use. An upper surface of the blade engages the epiglottis of a patient during use to reduce movement of the epiglottis and/or improve the visual field of the user.

Further, a surface finish on portions of the laryngoscope may reduce the reflectivity of the laryngoscope during use. This may enhance the direct visual field of the user, that is, the user may have a clearer view of the pharyngeal area.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts a schematic of a laryngoscope having a curved MacIntosh blade during use;

FIG. 2 depicts a schematic of a laryngoscope having a straight Miller blade during use;

FIG. 3 illustrates a side view of a laryngoscope having a straight Miller blade;

FIG. 4 illustrates a top view of the laryngoscope having a straight Miller blade;

FIG. 5 illustrates a bottom view of the laryngoscope shown having a straight Miller blade;

FIG. 6 illustrates a front perspective view of a laryngoscope having a straight Miller blade;

FIG. 7 illustrates a rear perspective view of a laryngoscope having a straight Miller blade;

FIG. 8 illustrates a side perspective view of a laryngoscope having a straight Miller blade;

FIG. 9 illustrates a side view of a laryngoscope having a straight Miller blade;

FIG. 10 illustrates a side view of a laryngoscope having a straight Miller blade;

FIG. 11 illustrates a front perspective view of a left part of a laryngoscope having a straight Miller blade;

FIG. 12 illustrates a bottom perspective view of a left part of a laryngoscope having a straight Miller blade;

FIG. 13 illustrates a bottom perspective view of a laryngoscope having a straight Miller blade and a handle;

FIG. 14 illustrates a front perspective view of a portion of a laryngoscope having a straight Miller blade;

FIG. 15 illustrates a bottom perspective view of a laryngoscope having a straight Miller blade and a handle;

FIG. 16 illustrates an enlarged bottom perspective view of a distal portion of a straight blade of a left part of a laryngoscope;

FIG. 17 illustrates a left side perspective view of a right part of laryngoscope having a straight Miller blade and a handle integrally formed;

FIG. 18 illustrates a left side perspective view of a right part of laryngoscope having a straight Miller blade and a handle integrally formed;

FIG. 19 illustrates a left side bottom perspective view of a right part of laryngoscope having a straight Miller blade and a handle integrally formed;

FIG. 20 depicts an image of intubation procedure using a laryngoscope having glossy surfaces and showing a reflection of the surrounding tissues;

FIG. 21 depicts an image of intubation procedure using a laryngoscope having glossy surfaces and showing a reflection of the surrounding tissues;

FIG. 22 illustrates a side view of a laryngoscope having a straight Miller blade;

FIG. 23 illustrates a side perspective view of a laryngoscope having a straight Miller blade;

FIG. 24 illustrates a bottom view of a laryngoscope having a straight Miller blade;

FIG. 25 illustrates a rear perspective view of a laryngoscope having a straight Miller blade;

FIG. 26 illustrates a side perspective view of a laryngoscope having a straight Miller blade;

FIG. 27 illustrates a top view of a laryngoscope having a straight Miller blade;

FIG. 28 illustrates a side view of a laryngoscope having a straight Miller blade;

FIG. 29 illustrates a side perspective view of a laryngoscope having a straight Miller blade;

FIG. 30 illustrates a bottom perspective view of a laryngoscope having a straight Miller blade;

FIG. 31 illustrates a bottom view of a laryngoscope having a straight Miller blade; and

FIG. 32 illustrates a top view of a laryngoscope having a straight Miller blade.

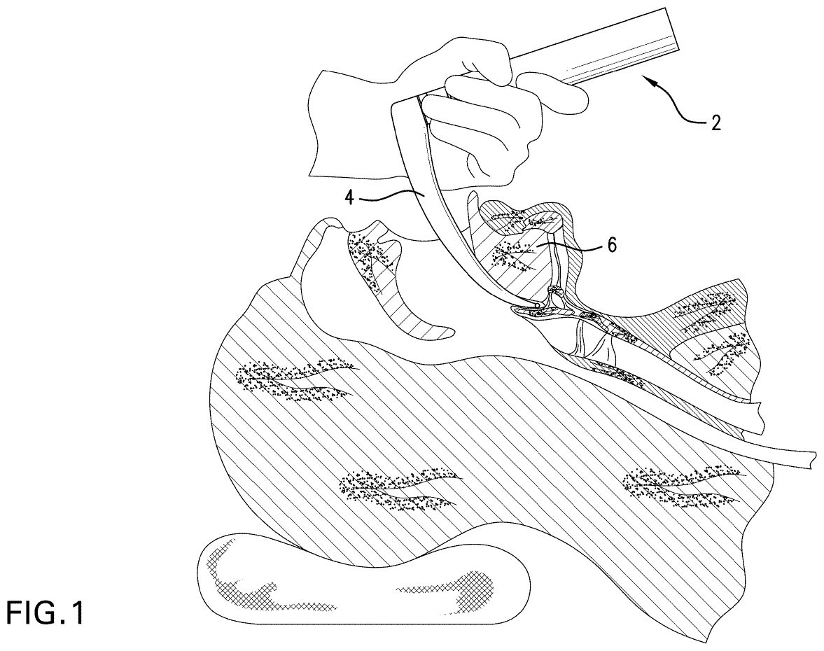

### DETAILED DESCRIPTION OF THE INVENTION

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments. In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship.

The present disclosure includes embodiments of a laryngoscope that may be configured to examine and/or clear the mouth and throat of a patient during medical procedures, for example, anesthesia, respiratory failure, etc.

Exemplary aspects of the laryngoscope include finishes that may be applied, molded into, etc. elements of the laryngoscope such that slippage is inhibited but the ease of entry of the blade into the mouth and/or the pharyngeal area of patients is maintained. Reducing and/or inhibiting slippage during use of laryngoscopes may be accomplished by controlling properties of the surfaces of laryngoscope. Surface finishes may be molded into elements of laryngoscope as they are formed and/or created on a finished element using etching, blasting, sparking, and/or application of coatings, films, foils, or the like.

Surface finishes as described herein may be defined by the Verein Deutscher Ingenieure (VDI) 3400 Standard. The VDI 3400 standard is a German standard that classifies surface roughness of materials into specific grades. In particular, the VDI 3400 Standard defines a series of roughness grades from 0 to 45. Each grade specifies a particular range of surface characteristics, in particular, an average roughness (Ra). For example, using the VDI 3400 standard a grade value equal to zero corresponds to an average roughness (Ra) of 0.10 μm and a grade value of 45 corresponds to an average roughness (Ra) of 18.00 μm.

Surface finishes may be applied to various surfaces, faces, and/or elements of the laryngoscope. In some instances, the surface finish may be applied to portions of the surfaces, faces, and/or elements of the laryngoscope. Surface finishes may include patterning of the finish including but not limited to shapes such as chevrons, polygons, rounded forms, parallel lines, intersecting lines, random spots, etc. and combinations thereof.

Various surfaces, faces, and/or elements of the laryngoscopes described herein may have surface finishes having a VDI 3400 Standard grade value in a range from about 15 to about 33 corresponding to average roughness values (Ra) in a range from about 0.56 μm to about 4.5 μm. Surface finishes having a VDI 3400 Standard grade value in a range from about 15 to about 30 may be used on various surface, faces, and/or elements of the laryngoscope corresponding to average roughness values (Ra) in a range from about 0.56 μm to about 3.15 μm. In some instances, a specific embodiment may include surfaces having a VDI 3400 Standard grade value in a range from about 18 to about 30 may be used on various surface, faces, and/or elements of the laryngoscope corresponding to average roughness values (Ra) in a range from about 0.8 μm to about 3.15 μm.

In some instances, surface finishes having a VDI 3400 Standard grade value in a range from about 18 to about 27 may be used on various surface, faces, and/or elements of the laryngoscope corresponding to average roughness values (Ra) in a range from about 0.8 μm to about 2.24 μm. In an embodiment, it may be desired to use surface finishes having a VDI 3400 Standard grade value in a range from about 21 to about 24 on various surface, faces, and/or elements of the laryngoscope corresponding to average roughness values (Ra) in a range from about 1.12 μm to about 1.60 μm.

In a specific embodiment, a laryngoscope may include surface finishes having a VDI 3400 Standard grade value in a range from about 27 to about 30 on various surface, faces, and/or elements of the laryngoscope corresponding to average roughness values (Ra) in a range from about 2.2 μm to about 3.15 μm.

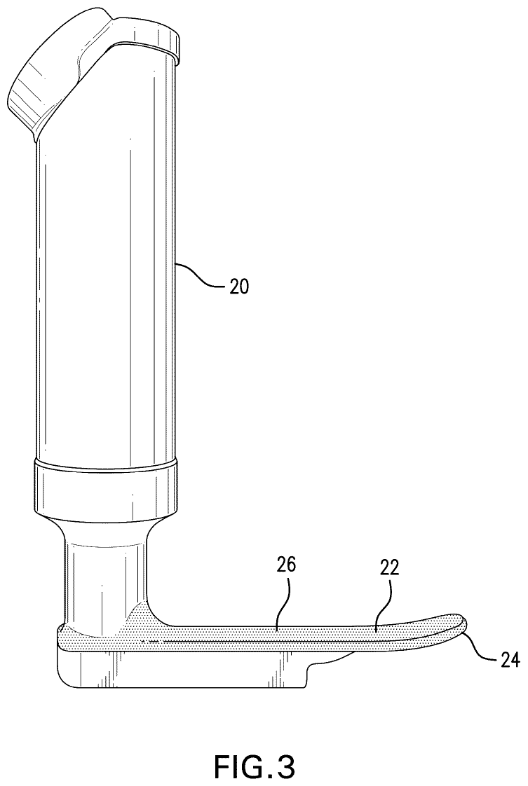

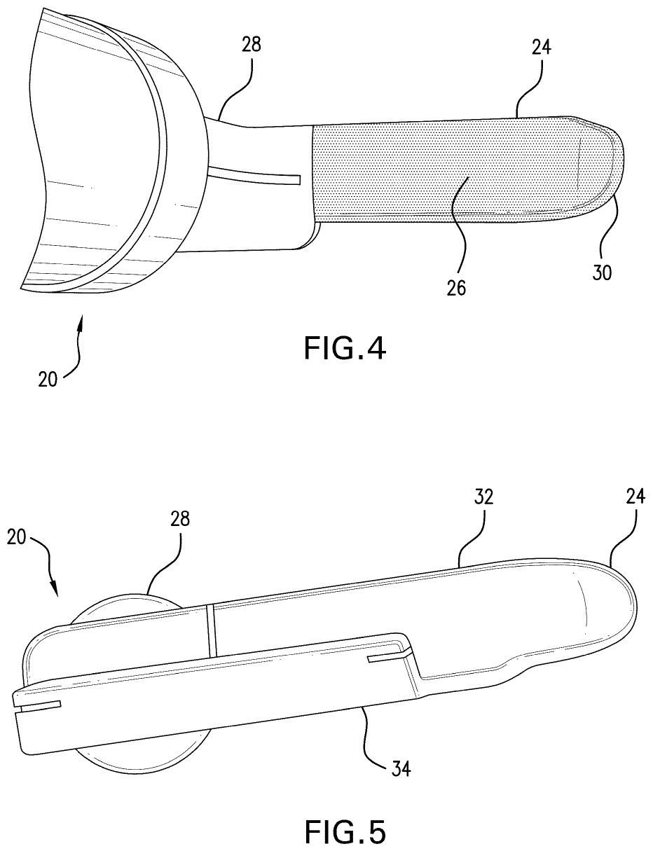

Surface finishes may extend across a predetermined length or a predetermined area of the surface of a blade. In some instances, a surface finish may extend across an entire face of the laryngoscope. Referring now to the figures, FIG. 3 illustrates a side view of a laryngoscope 20 having an upper surface 22 of blade 24 that includes a surface finish 26. As shown in FIG. 4, the surface finish 26 on the upper surface 22 of the blade 24 extends from the handle 28 to the distal end 30 of the blade.

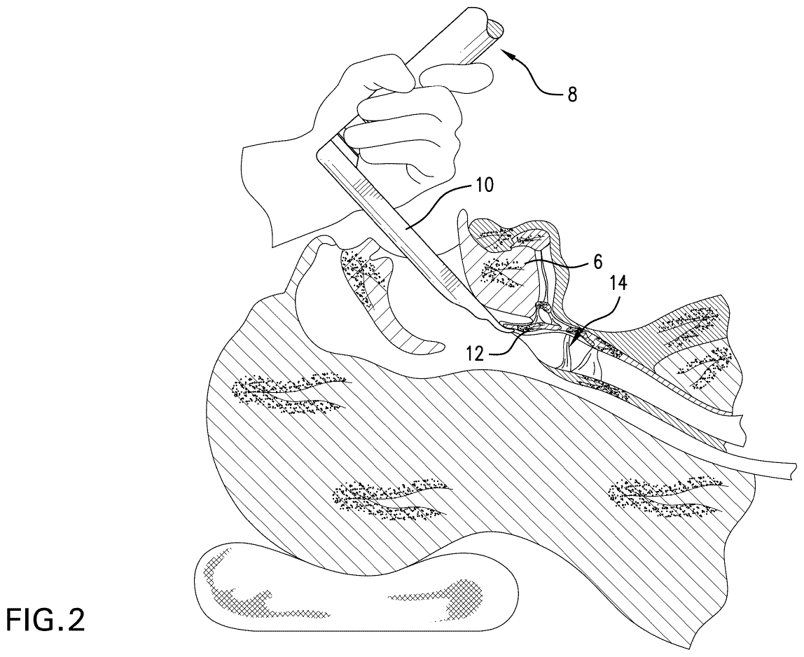

Surface finishes may be positioned on one or more portions, surfaces, and/or elements. Surface finishes may be selectively positioned on surfaces of a laryngoscope to impart specific characteristics to specific portion of the laryngoscope. For example, in some instances a laryngoscope may have specific portions having predetermined values for a characteristic such as a coefficient of friction and/or a reflectivity. As shown in FIG. 4, a surface finish 26 extends a predetermined length of the upper surface 22 of the blade 24 from the handle 28 to the distal end 30. During use the distal end may engage soft tissues of the patient, for example, an epiglottis. The surface finish on the distal end may engage the epiglottis in a manner such that the epiglottis is moved up towards the tongue as shown in FIG. 2.

As shown in FIGS. 3-4, an upper surface 22 includes surface finish 26 in a range from about fifteen (15) to about thirty (30) when measured on the VDI 3400 Standard scale. For example, surface finishes of an upper surface of a laryngoscope may have a grade value in a range from about eighteen (18) to about thirty (30) when classified using the VDI 3400 Standard scale. In some instances, the surface finishes of the upper surface may have a grade value in a range from about eighteen (18) to about twenty-four (24) when classified using the VDI 3400 Standard scale. In a particular example of a laryngoscope having a substantially straight blade, may have a surface finish on the upper surface that has a grade value substantially within a range from about twenty-one (21) to about twenty-four (24) when classified using the VDI 3400 Standard scale. In a specific example of a laryngoscope having a substantially straight blade, a surface finish on the upper surface may have a grade value substantially within a range from about twenty-seven (27) to about thirty (30) when classified using the VDI 3400 Standard scale. In alternate embodiments, the surface finish may be limited to the tip of the blade that will contact the epiglottis.

In some embodiments, the surface finish shown in FIG. 4 may be formed during molding. For example, in a particular embodiment a surface finish may be formed using molds used to form parts of a laryngoscope. In particular, a mold may include a texture that is imparted to predetermined portions, surfaces, and/or faces of the laryngoscope as a surface finish. Thus, during the molding process molds may be constructed such that areas of the mold corresponding to areas of the laryngoscope that have a surface finish are textured. In some embodiments, materials may affect a texture necessary in a mold to achieve a predetermined surface finish on a portion of a laryngoscope.

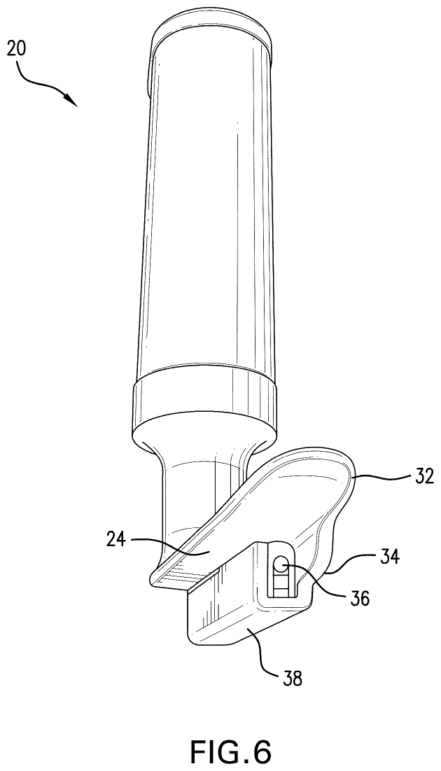

FIGS. 5-8 depict various additional views of the laryngoscope of FIGS. 3-4 having multiple surfaces where the majority of the surfaces do not include a surface finish. FIG. 5 illustrates a view of an underside 32 of blade 24 of laryngoscope 20 that includes projection 34 extending from the underside 32. As shown in FIG. 6, blade 24 includes projection 34 on a portion of the underside 32. Projection 34 includes optical element 36. As shown projection 34 runs along a portion of the underside 32 of blade 24. In some embodiments, an optical element may include a camera, a light element, a lens, and/or a combination thereof.

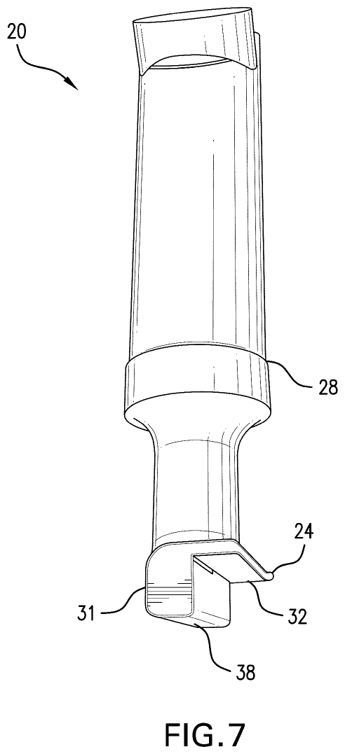

A further rear perspective view of laryngoscope 20 is shown in FIG. 7. Laryngoscope 20 includes blade 24 and handle 28. Blade 24 has projection 34 positioned on a portion of the underside 32. Projection 34 includes a lower surface 38.

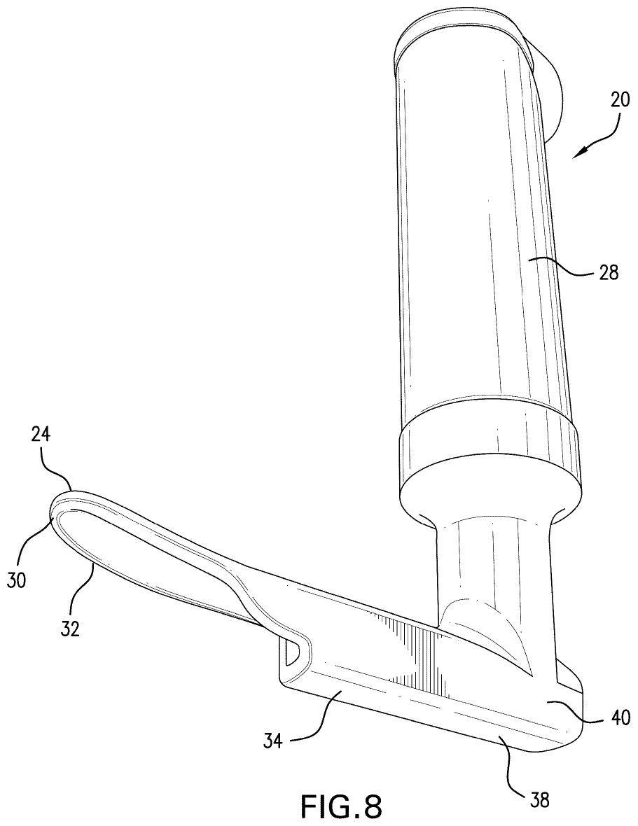

FIG. 8 illustrates a side perspective view of a laryngoscope 20 that includes blade 24 and handle 28. Projection 34 extends only a portion of blade 24. Sidewall 40 extends beyond projection 34 couples to the upper surface 22 of blade 24.

In some embodiments, an angled section may include a surface finish. The surface finish may be on an upper surface of the blade. Alternatively, the surface finish may be on both the upper surface and lower surface of the angled section. In some embodiments, the entire angled section may include a surface finish.

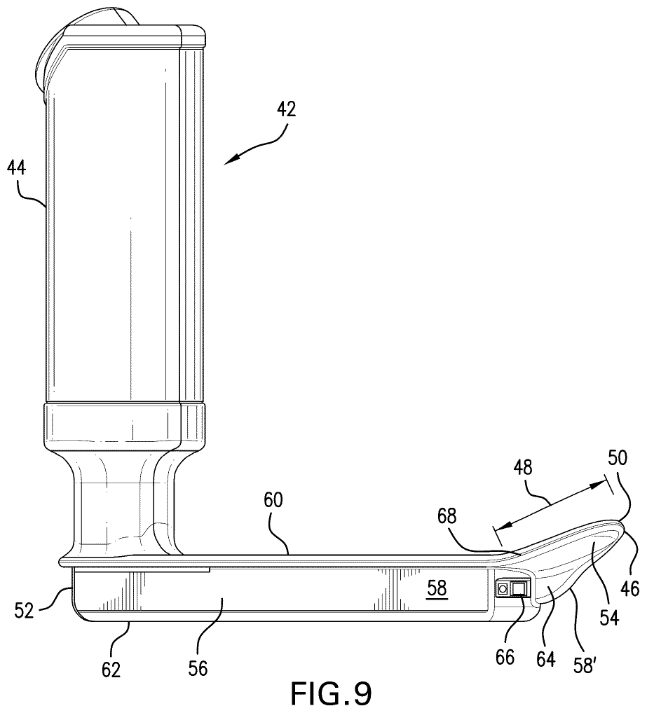

FIG. 9 illustrates a right side perspective view of an embodiment of a laryngoscope 42. Laryngoscope 42 includes handle 44 and blade 46. Blade 46 includes angled portion 48 proximate distal end 50 of the laryngoscope. Projection 56 runs from a proximal end 52 of the blade 46 to the angled portion 48 at the distal end 50 of the blade. Underside 54 of the angled portion 48 includes an extension 64 running between angled portion 48 and projection 56 and acting as part of left sidewall 58′. A surface finish 68 is positioned on an upper surface 60 of the angled portion 48 of the blade 46.

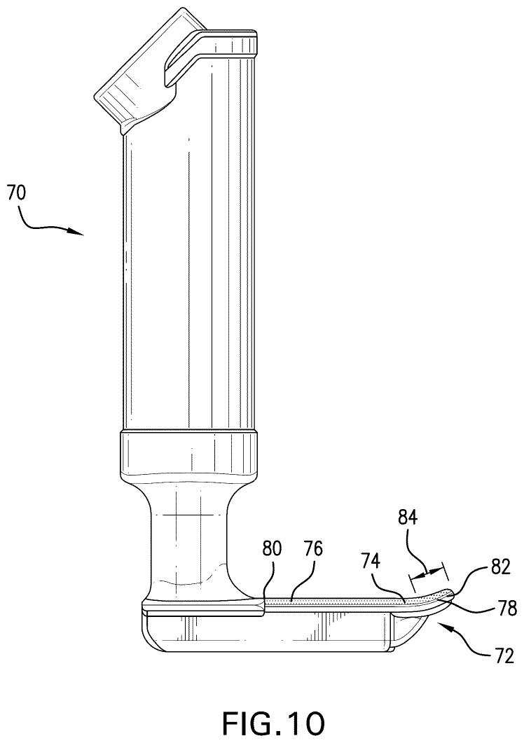

In some embodiments, a laryngoscope 70 includes a blade 72 having a surface finish 74 on an upper surface 76 and edge 78 as shown in FIG. 10. Laryngoscope 70 as illustrated in FIG. 10 includes a blade 72 having a proximal end 80 and a distal end 82 that includes angled section 84.

A surface finish may be positioned on a portion of a laryngoscope such that slippage is inhibited during use is inhibited. For example, as shown in FIG. 10 a surface finish may be applied to an angled portion of a blade on both an upper surface and an edge such that the surface finish engages soft tissues, for example, an epiglottis during use. However, ease of entry of the blade into the mouth and/or the pharyngeal area of patient's needs to be maintained while reducing slippage. Thus, determining a type of surface finish that reduces and/or inhibits slippage when positioned in the working position in the pharyngeal area and yet allows for movement of the laryngoscope through the mouth and into the pharyngeal area is critical.

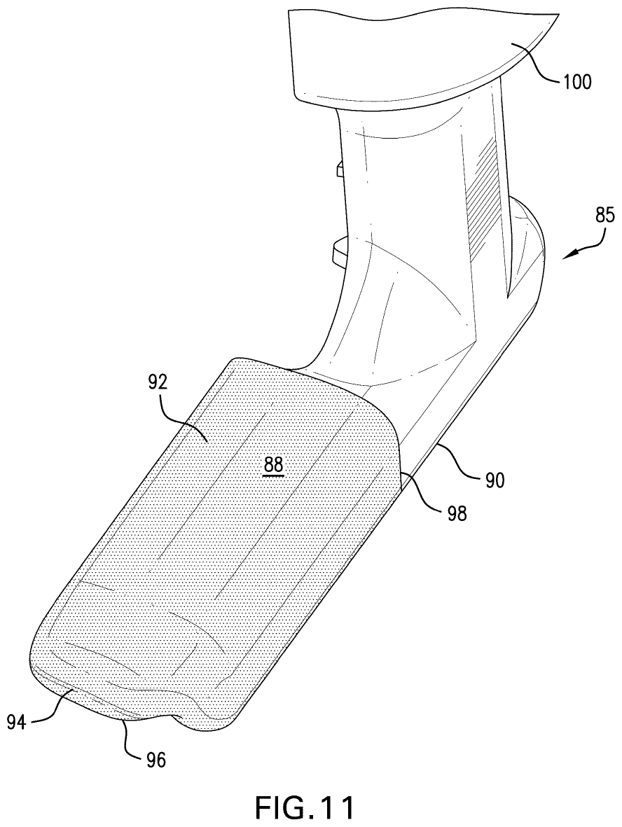

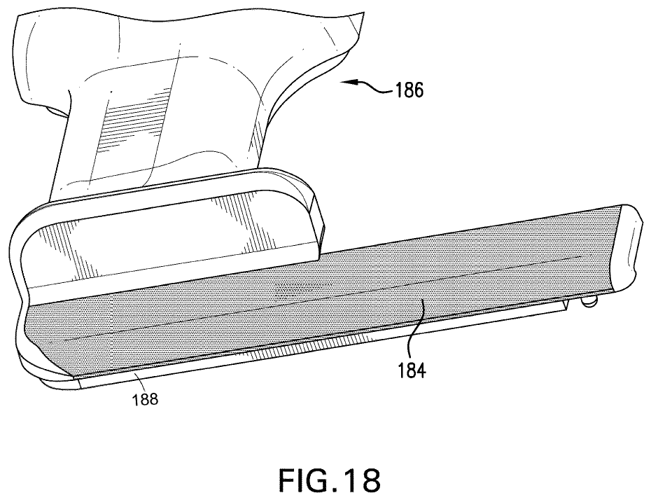

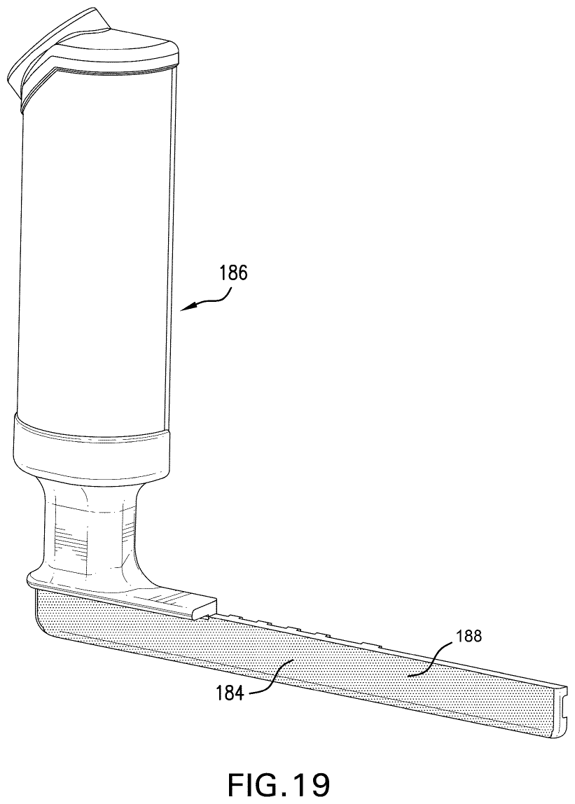

FIGS. 11-12 and 17-19 depict separate parts (i.e., left and right parts) that are formed and combined to create a laryngoscope. Parts may be formed by three dimensional printing such as Vat polymerization, powder bed fusion (e.g., laser sintering), material jetting, binder jetting, directed energy deposition, molding such as injection molding, CNC machining, casting, or a combination thereof.

The parts shown in FIGS. 11-19 are the result of an injection molding process. Materials that may be used in a laryngoscope may include any plastics known in the art for use in medical devices. Some embodiments may include use of different materials for different elements of the laryngoscope. In some embodiments, laryngoscopes may be injection molded from plastics such as acrylonitrile butadiene styrene (ABS) or polycarbonate (PC). In a particular embodiment, a material such as ABS or PC may be used for a majority of the laryngoscope or the entire laryngoscope. For example, a laryngoscope may be made from acrylonitrile butadiene styrene (ABS). Utilizing a single material may for a laryngoscope may be cost effective.

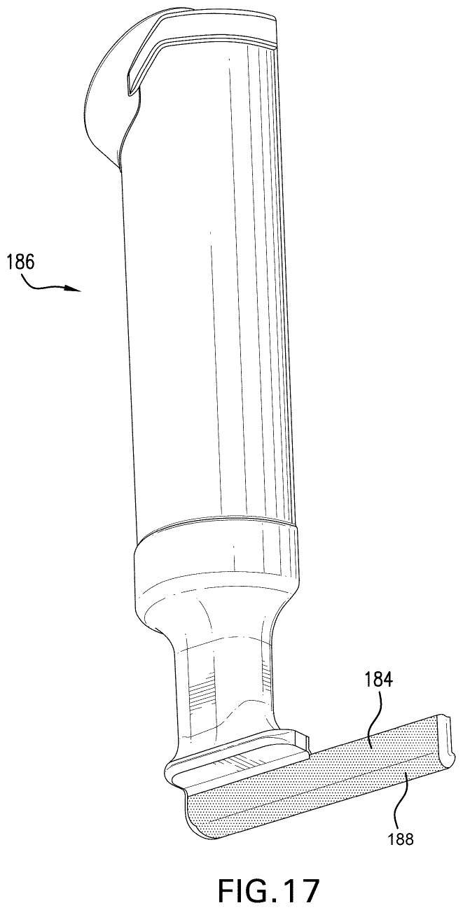

FIGS. 11-16 depict various left parts 85 of a laryngoscope while FIGS. 17-19 depict different right parts 186 of a laryngoscope. As is shown in FIGS. 11-19, each part includes a blade portion and a handle portion.

A front perspective view of a left part of laryngoscope having a straight Miller blade and a handle integrally formed is shown in FIG. 11. An upper surface 88 of blade 90 includes a surface finish 92 corresponding to the shaded section. Thus, the surface finish 92 extends from a tip 94 of a distal end 96 to a line 98 proximate the handle 100. Further, the surface finish extends around the blade 90.

Some embodiments may include a substantially consistent surface finish across all surfaces of a laryngoscope having a surface finish. For example, external surfaces of a blade may have a grade value substantially within a range from about eighteen (18) to about twenty-four (24) when classified using the VDI 3400 Standard scale. In a particular instance, all of the surface finishes of a laryngoscope may include a surface finish having a grade value in a range from about 21 to about 24 according to the VDI 3400 standard. Some embodiments of a laryngoscope that have a substantially straight blade may have surface finishes on all surfaces of the laryngoscope having a grade value substantially within a range from about twenty-seven (27) to about thirty (30) when classified using the VDI 3400 Standard scale.

In some embodiments, multiple areas on a laryngoscope may include differing surface finishes. For example, a distal end of the upper surface of a blade may have a surface finish grade value in a range from about 21 to about 24 according the VDI 3400 standard while other portions of the blade may have a surface finish in a range from about 18 to about 21 according to the VDI 3400 standard. In a particular embodiment, a distal end of an upper surface of a blade may have finish grade value in a range from about 27 to about 30 according to the VDI 3400 standard while other portions of the blade may have a surface in a range from about 18 to about 24 according to the VDI 3400 standard.

A medical professional may select a laryngoscope for use in a procedure based on a predetermined surface finish grade value when evaluated using the VDI 3400 standard and/or a predetermined surface configuration. In some embodiments, one or more surfaces of a laryngoscope may have a surface finish in a range from about fifteen (15) to about thirty (30) when measured on the VDI 3400 Standard scale. Some uses may require the use of a laryngoscope having a surface finish on one or more surfaces with a grade value in a range from about eighteen (18) to about twenty-four (24) when classified using the VDI 3400 Standard scale. For example, in a particular instances one or more surfaces on a laryngoscope may have surface finish grade value defined by the VDI 3400 standard in a range from about 21 to about 24.

Some uses may require the use of a laryngoscope having a surface finish on one or more surfaces with a grade value in a range from about eighteen (18) to about thirty (30) when classified using the VDI 3400 Standard scale. In a further example, one or more surfaces on a laryngoscope may have a surface finish grade value defined by the VDI 3400 standard in a range from about 27 to about 30.

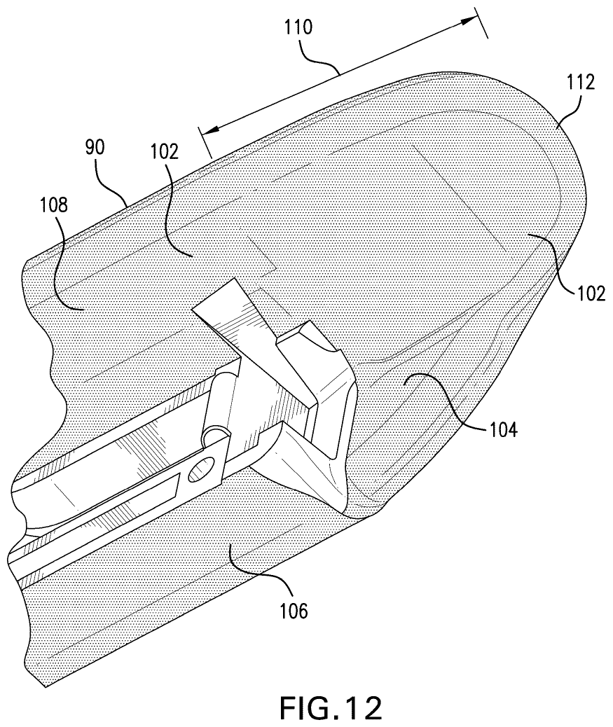

FIG. 12 illustrates an enlarged bottom right perspective view of the left part of the laryngoscope shown in FIG. 11. As is shown in FIG. 12 the surface finish extends around the blade 90 to cover underside blade portion 102, sidewall 104, left part bottom section 106, and including ledge 108, angled portion 110, and tip 112.

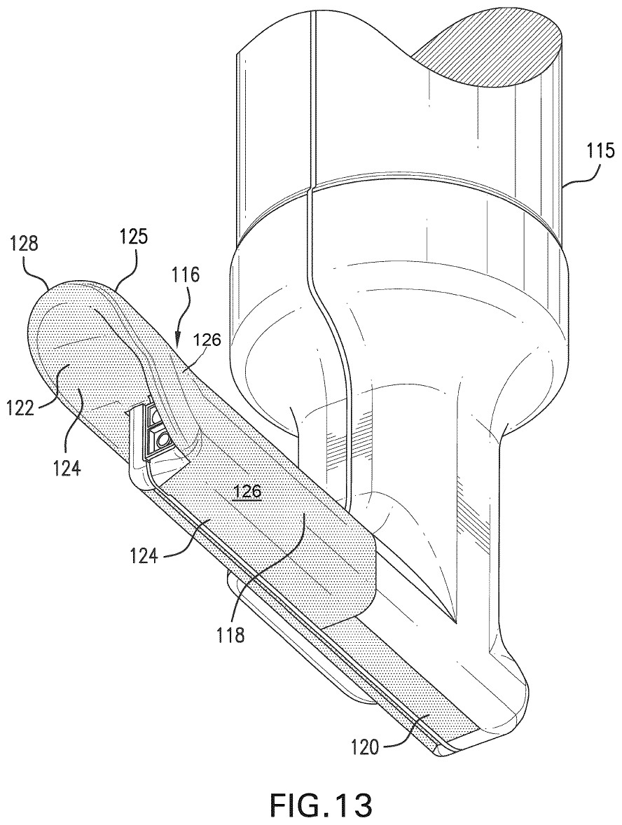

FIG. 13 illustrates an enlarged bottom left perspective view of the laryngoscope 115 shown in FIGS. 11-12. As is shown in FIG. 13 the surface finish 124 (shown as a shaded portion 126) extends around the blade 116 to cover sidewall 118, left part bottom section 120, and underside blade portion 122 including angled portion 125 and tip 128.

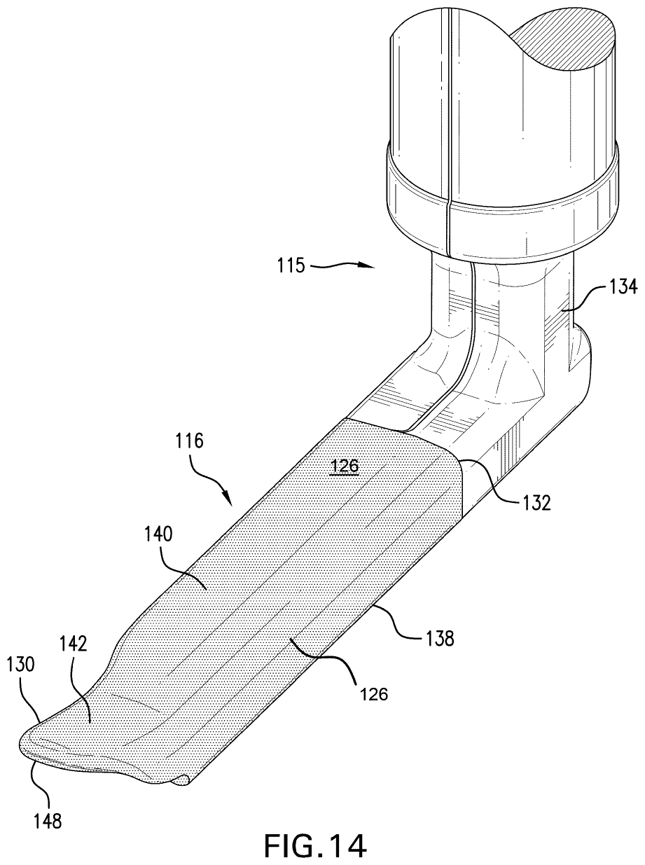

A further embodiment of a laryngoscope is depicted in FIG. 14. Blade 116 has a longer configuration here. Surfaces 138, 140, 142 of blade 116 include a surface finish 124 shown as shaded section 126. Thus, the surface finish 124 extends from the tip 148 of the distal end 130 to the line 132 proximate the handle portion 134. Further, the surface finish extends around the blade 116.

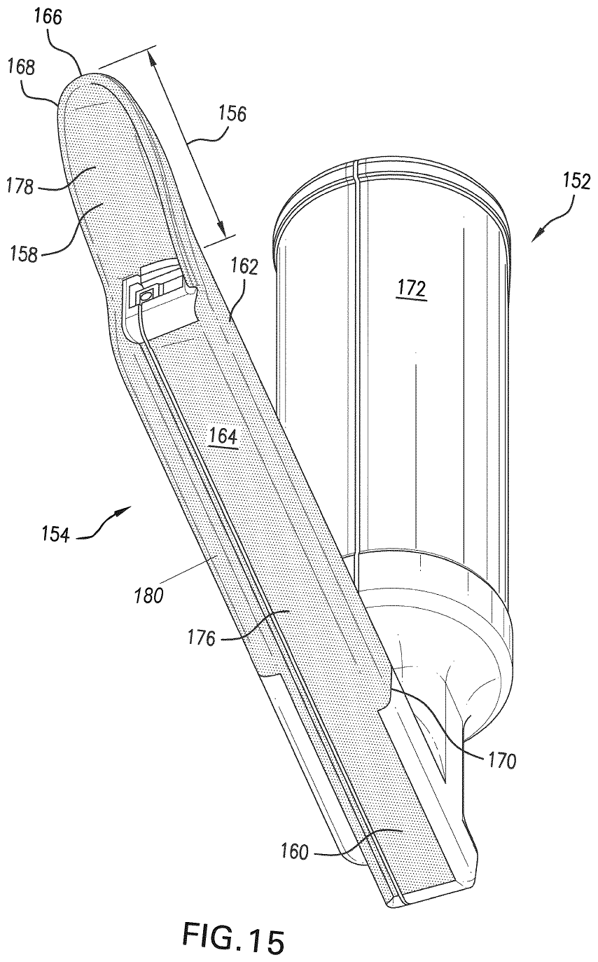

A further embodiment of a laryngoscope 152 is depicted in FIG. 15. Blade 154 has a longer configuration here and includes an angled portion 156. Surfaces 158, 160, 162 of blade 154 include surface finishes 164 (shown as shaded section). Thus, the surface finish 164 extends from the tip 166 of the distal end 168 to the line 170 proximate the handle portion 172 on an upper surface of the blade. Further, the surface finish extends around the blade 154. Thus, surface finish 164 extends from an upper surface around the blade to cover sidewall 174, left part bottom section 176, and underside blade portion 178 including ledge 180, angled portion 156, and tip 166 include a predetermined surface finish 164 as shown by the shaded areas of FIG. 15.

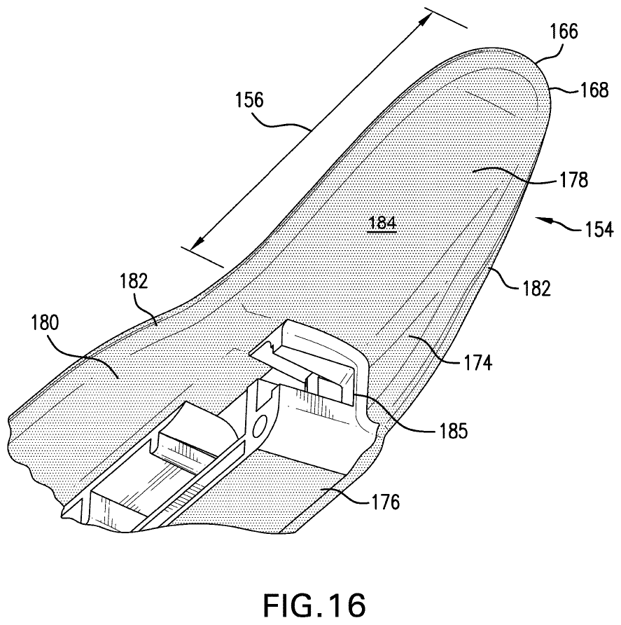

FIG. 16 illustrates an enlarged bottom right perspective view of the left part of the laryngoscope shown in FIG. 15. As is shown in FIG. 16 the surface finish extends around the blade 154 to cover sidewall 174, left part bottom section 176, and underside blade portion 178 including ledge 180, angled portion 156, and tip 166. As is illustrated in FIG. 16, edges 182 of the various faces includes surface finish 184 (depicted as shading). As shown in FIG. 16, surfaces that are not shaded do not include a surface finish. In some instances, such surfaces may be polished. For example, surfaces proximate opening 185 do not include a surface finish. In particular, surfaces proximate the opening for the camera may be polished. In particular, surfaces without a surface finish may be polished such that they have a predetermined SPI/SPE grading.

FIGS. 17-19 depict various views of the surface finish 184 (e.g., shaded sections) on blade portion 188 of a right part 186 of a laryngoscope.

During use an assembled laryngoscope having predetermined areas of surface finish may be inserted into the mouth. As discussed above, use of a substantially straight blade similar to a Miller blade entails using a distal end of the blade to secure the epiglottis as depicted in FIG. 2. As known in the field, the Miller blade technique involves securing at a least a portion of the epiglottis with the blade tip. Use of a predetermined surface finish on the elements of the laryngoscope contacting the epiglottis may reduce and/or inhibit slippage of the laryngoscope during use.

External surfaces of a blade of a laryngoscope may have a grade value substantially within a range from about eighteen (18) to about thirty (30) when classified using the VDI 3400 Standard scale.

In a particular example, external surfaces of a blade may have a grade value substantially within a range from about eighteen (18) to about twenty-four (24) when classified using the VDI 3400 Standard scale.

External surfaces of a blade may have a grade value substantially within a range from about twenty-one (21) to about twenty-four (24) when classified using the VDI 3400 Standard scale.

In a further example, external surfaces of a blade may have a grade value substantially within a range from about twenty-seven (27) to about thirty (30) according to the VDI 3400 Standard scale.

In a particular embodiment, an angled portion of a blade may include a surface finish on an upper surface only. For example, an angled portion of a blade may have a grade value substantially within a range from about eighteen (18) to about twenty-four (24) when classified using the VDI 3400 Standard scale.

Some embodiments may include an angled portion of a blade having a grade value substantially within a range from about twenty-one (21) to about twenty-four (24) when classified using the VDI 3400 Standard scale.

In a further embodiment, an angled portion of a blade may have a grade value substantially within a range from about twenty-seven (27) to about thirty (30) according to the VDI 3400 Standard scale.

In some embodiments, an area of the laryngoscope that comes into contact with the patient's tongue may also include a surface finish to reduce and/or inhibit slippage of the laryngoscope. However, surface finishes used on portions of the laryngoscope may be limited to particular VDI 3400 Standard grade values having a predetermined average roughness to reduce or inhibit the laryngoscope's ability to damage tissues of the patient.

In a particular embodiment, a matte finish may be used on a majority of the surfaces of the blade of a laryngoscope. A matte finish may allow a better visual during use. For example, a matte finish on the blade may reduce and or inhibit glare in the field of vision of a user and/or a camera. In particular, a matte finish on portions of the blade may have a grade value in a range from about 15 to about 33 on the VDI 3400 standard corresponding to an average roughness values (Ra) in a range from about 0.56 μm to about 4.5 μm. For example, a matte finish on the blade may have a grade value in a range from about 15 to about 30 on the VDI 3400 standard corresponding to an average roughness values (Ra) in a range from about 0.56 μm to about 3.15 μm. In a further example, a matte finish on a blade of a laryngoscope may include a surface finish having a VDI 3400 Standard grade value in a range from about 18 to about 30 may be used corresponding to average roughness values (Ra) in a range from about 0.8 μm to about 3.15 μm.

In some instances, surface finishes having a VDI 3400 Standard grade value in a range from about 18 to about 27 may be used on elements of the laryngoscope corresponding to average roughness values (Ra) in a range from about 0.8 μm to about 2.24 μm.

In an embodiment, it may be desired to use surface finishes having a VDI 3400 Standard grade value in a range from about 21 to about 24 on various elements of the laryngoscope corresponding to average roughness values (Ra) in a range from about 1.12 μm to about 1.60 μm.

In a further embodiment, it may be desired to use surface finishes having a VDI 3400 Standard grade value in a range from about 27 to about 30 on various elements of the laryngoscope corresponding to average roughness values (Ra) in a range from about 2.2 μm to about 3.15 μm.

One or more surfaces on a blade of a laryngoscope may include surface finishes that are configured to non-traumatically engage soft tissues of a patient during use. In some instances, surface finishes on an upper surface of a blade may engage a portion of a patient's epiglottis and/or tongue when the laryngoscope is inserted into the patient's mouth. The upper surface of the blade may include a surface finish that engages the patient's soft tissues, for example, the epiglottis and/or tongue to help prevent or resist movement of the epiglottis and/or tongue.

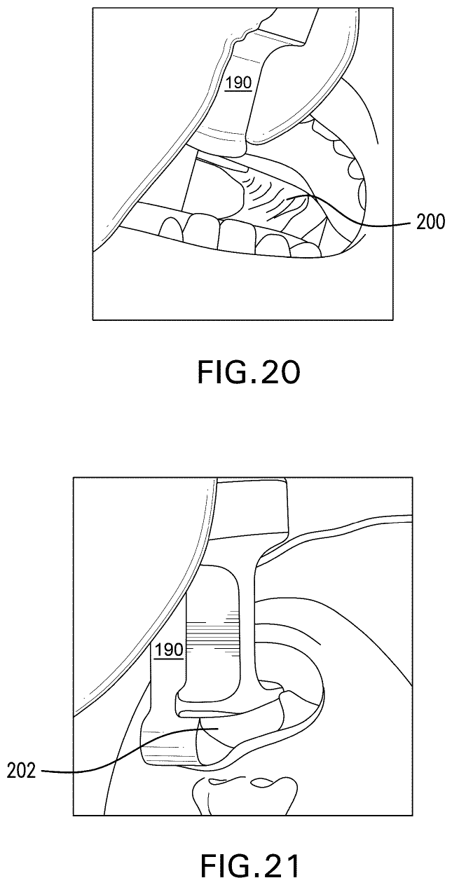

In some instances, surfaces on a blade may include a surface finish that reduces the reflectivity of the surface during use. Reducing the reflectivity of surfaces of a laryngoscope may provide a user with a better field of vision during use of the laryngoscope. By reducing reflectivity of the material of the laryngoscope glare and/or reflections of tissue may be reduced thereby improving the field of vision for the user. FIGS. 20-21 show images of procedures using a standard gloss finish for a laryngoscope 190 and illustrate reflections 200, 202 from surrounding tissues. These reflections may interfere with a user's view during an intubation.

Thus, reducing reflections and/or glare from surfaces of the laryngoscope may provide a user with a better view of the mouth and/or pharyngeal area during use. Further, reducing reflections and/or glare may improve images from any optical elements in use during a procedure.

In some instances, these surface finishes may be added to laryngoscopes having a straight blade (i.e., Miller Blade) or a curved blade (i.e., a MacIntosh blade or a hyperangulated blade).

As shown in FIG. 6 a laryngoscope 20 includes an optical device 36 positioned on underside 32 of blade 24. For example, using a surface finish 126 on surfaces of the blade 124 as illustrated in FIGS. 13-14 reduces reflections during use. In particular, the surface finishes of the lower surface may have a grade value in a range from about eighteen (18) to about twenty-four (24) when classified using the VDI 3400 Standard scale. In another embodiment, surface finishes of the lower surface of a laryngoscope may have value in a range from about eighteen (18) to about thirty (30) when classified using the VDI 3400 standard.

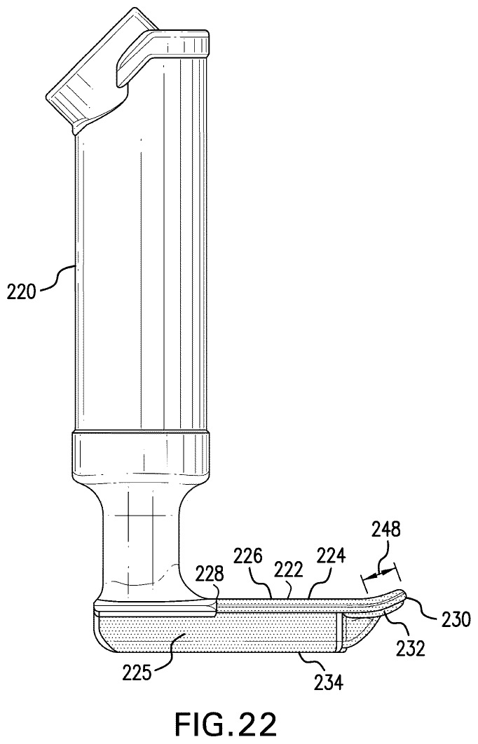

Examples of laryngoscope configurations are depicted in FIGS. 22-26 depict various views of the laryngoscope of having multiple surfaces that include a surface finish on multiple faces of the laryngoscope. As depicted in FIGS. 22-32 surfaces depicted as shaded are surfaces that include a surface finish. In particular, surfaces showing a surface finish in the examples (depicted in FIGS. 22-32 as a dotted surface) have a surface finish with a VDI 3400 Standard grade value in a range from about 21 to about 24. Thus, the elements shown in FIGS. 22-32 of the laryngoscope having a surface finish have average roughness values (Ra) in a range from about 1.12 μm to about 1.60 μm.

In an alternate embodiment of the images shown in FIGS. 22-32 surfaces depicted as shaded include a surface finish. In particular, surfaces showing a surface finish in the examples (depicted in FIGS. 22-32 as a dotted surface) may have a surface finish with a VDI 3400 Standard grade value in a range from about 27 to about 30. Thus, the elements shown in FIGS. 22-32 of the laryngoscope may have a surface finish have average roughness values (Ra) in a range from about 2.2 μm to about 3.15 μm.

Referring now to the figures, FIG. 22 illustrates a side view of a laryngoscope 220 having an upper surface 222 of blade 224 that includes a surface finish 226. The surface finish 226 on the blade 224 extends from a line proximate the handle 228 to the distal end 230 of the blade. Distal end 230 includes angled portion 248 having surface finish 226 on both the upper surface and underside 232 of the angled portion 248. Surface finish 226 extends alongside wall 225 of projection 234 as shown.

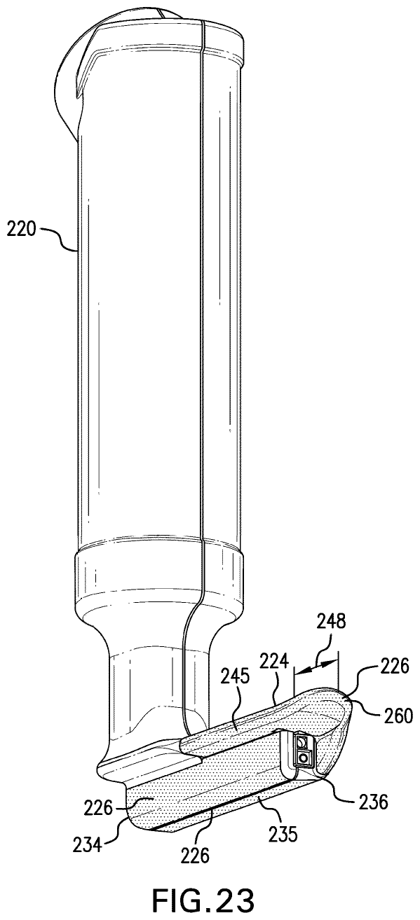

FIG. 23 depicts a side perspective view of laryngoscope 220 that includes blade 224 with projection 234 positioned on a portion of the underside of the blade. Housed in projection 234 is optical element 236. As shown projection 234 runs along a portion of the underside 232 of blade 224. Surface finishes are found on blade 224 on underside blade portion 232, sidewall 225, bottom section 235, and including ledge 245, angled portion 248, and tip 260.

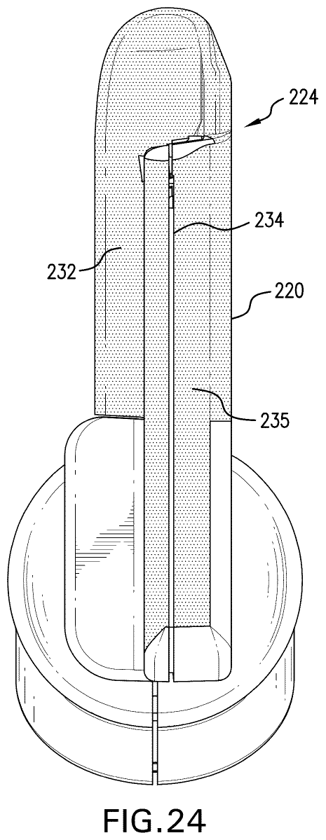

FIG. 24 illustrates a bottom view of laryngoscope 220 shown in FIGS. 22-23. As depicted in FIG. 24 an underside 232 of blade 224 of laryngoscope 220 includes projection 234 extending from the underside 232. As shown projection 234 runs along a portion of the underside 232 of blade 224. Portions of the projection 234 include surface finishes as depicted in FIG. 24. In particular, bottom section 235 of the blade includes a surface finish as shown.

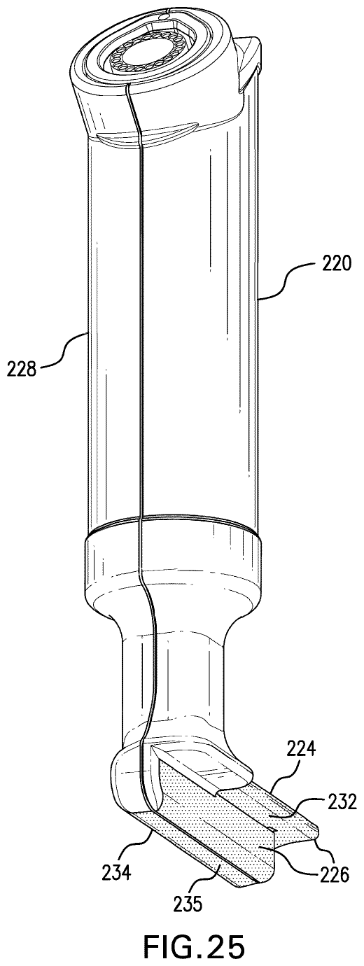

A rear perspective view of laryngoscope 220 is shown in FIG. 25. Laryngoscope 220 includes blade 224 and handle 228. Blade 224 has projection 234 positioned on a portion of the underside 232. Projection 234 includes a lower surface 238. The surface finish is depicted on various portions of the laryngoscope as indicated by the dots in FIG. 25.

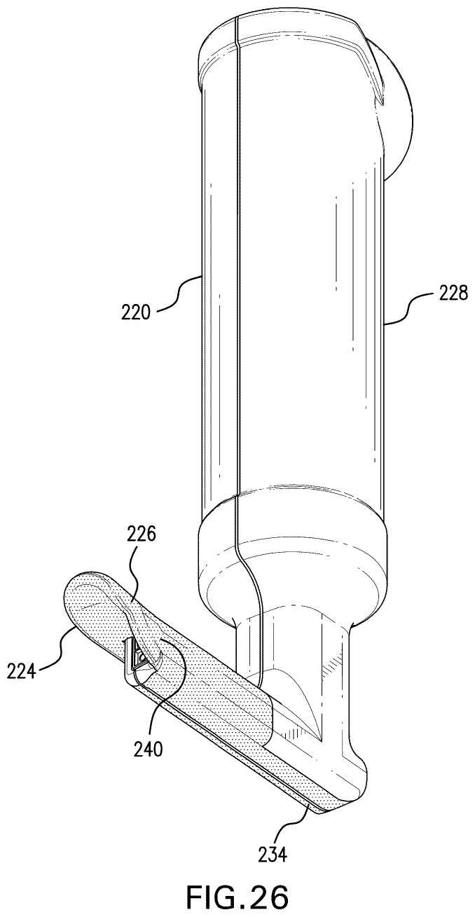

FIG. 26 illustrates a side perspective view of a laryngoscope 220 that includes blade 224 and handle 228. Projection 234 extends only a portion of blade 224. Sidewall 240 extends beyond projection 234 and couples to the upper surface 222 of blade 224. As indicated in FIG. 26, the surface finish extends from a tip of the blade to a line on the blade proximate the handle on the side wall of the laryngoscope.

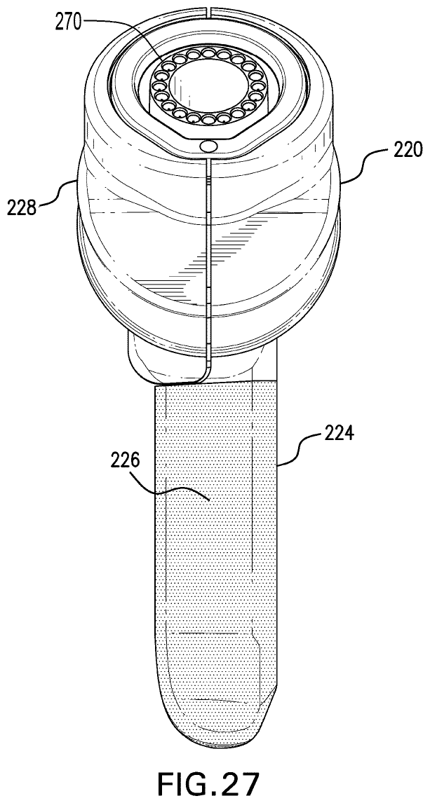

FIG. 27 illustrates a top perspective view of a laryngoscope 220 that includes blade 224 and handle 228. Handle 228 includes coupler 270 at a top surface. Surface finish 226 extends from a position proximate the handle to a tip of the blade.

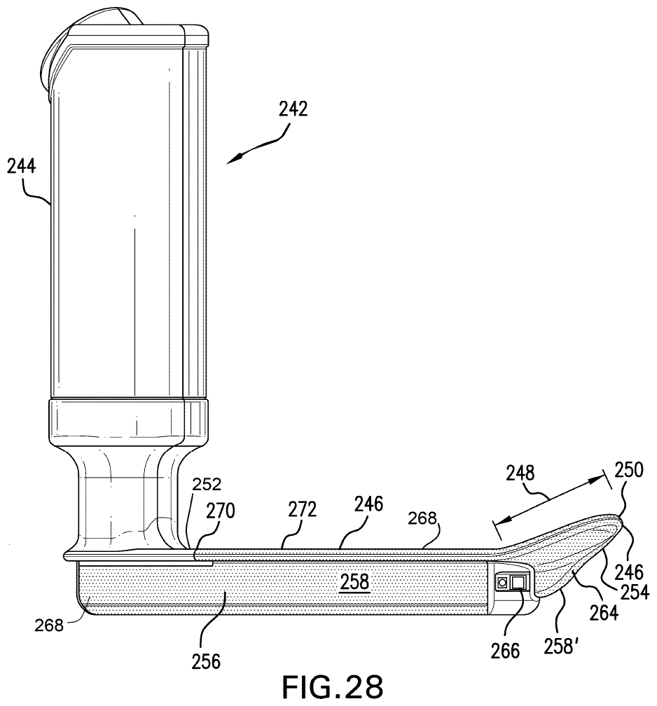

A right side perspective view of an example of a laryngoscope 242 is shown in FIG. 28. Laryngoscope 242 includes handle 244 and blade 246. Blade 246 includes angled portion 248 proximate distal end 250 of the laryngoscope. Projection 256 runs from a proximal end 252 of the blade 246 to the angled portion 248 at the distal end 250 of the blade. Underside 254 of the angled portion 248 includes an extension 264 running between angled portion 248 and projection 256 and acting as part of left sidewall 258′. As shown surface finish 268 is positioned multiple surfaces of the blade 246 and projection 256 as depicted by the shading. Thus, in this example the surface finish 268 extends from distal end 250 to line 270 on upper surface 272 of blade 246. Surface finish 268 extends the length of side wall 258 on projection 256 as shown. Further, the underside 254 of the angled portion 248 includes surface finish 268 as indicated by dots.

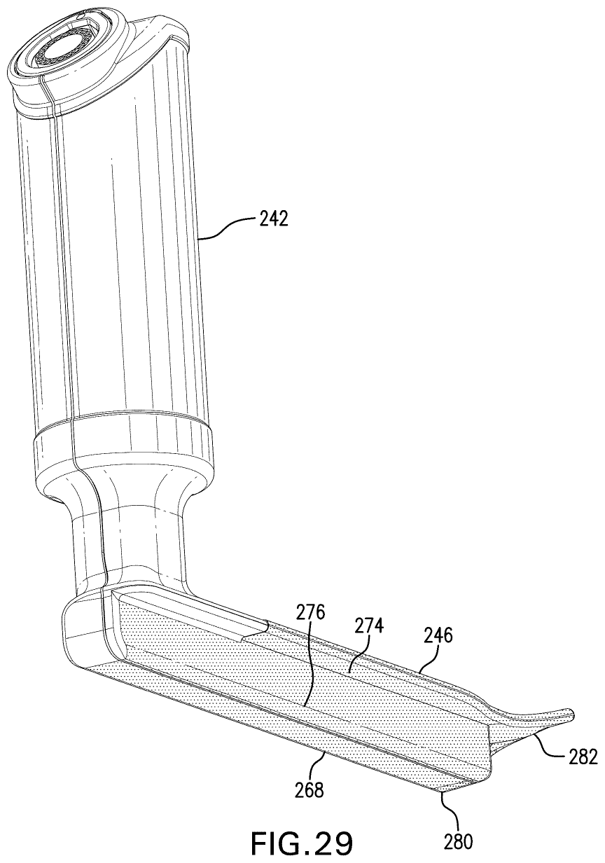

FIG. 29 is a rear perspective view of the laryngoscope 242 shown in FIG. 28. Surface finish 268 is present on multiple surfaces of the blade 246 as indicated by the shading. As illustrated in FIG. 29, the surface finish is present on ledge 274, side wall 276, bottom surface 280, and angled portion 282.

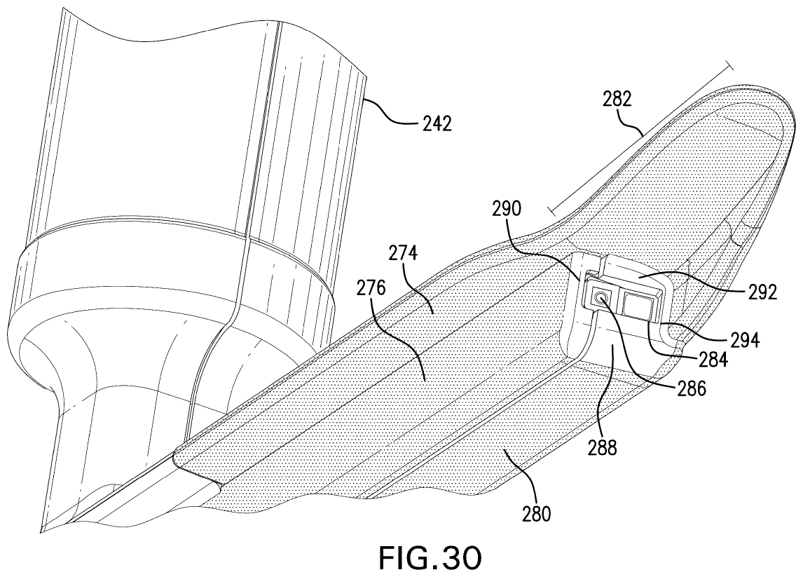

FIG. 30 shows an enlarged image of a bottom perspective view of laryngoscope 242. Underside of blade 246 includes surface finish 268 on ledge 274, side wall 276, bottom surface 280, and angled portion 282 as indicated by the dot structure. Blade 246 includes opening 284 for optical device 286. Surfaces 288, 290, 292, 294 proximate the opening 284 do not include the surface finish as described.

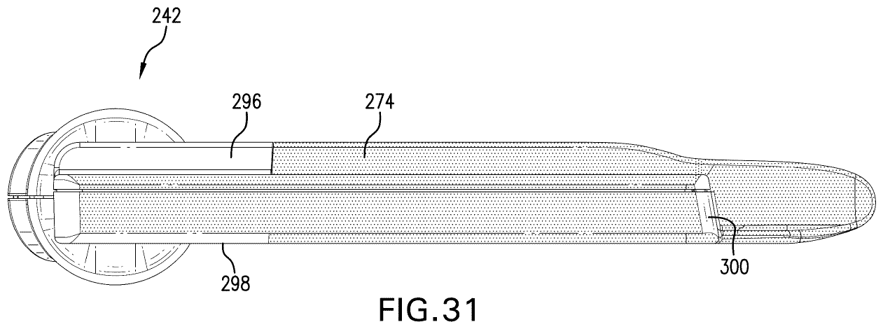

As shown in the example of a laryngoscope illustrated by FIG. 31, a surface finish 268 extends partially down the length of blade 246. Thus, portion 296 of ledge 274 and portion 298 of left side of blade do not include a surface finish as described herein. Further, portion 300 which is proximate the opening for the optical device does not include the surface finish described herein.

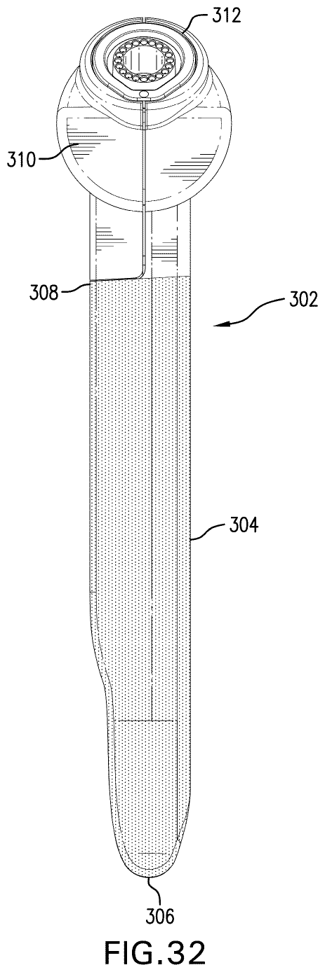

FIG. 32 depicts a top view of an example of laryngoscope 302. Surface finish 304 extends from distal end 306 to line 308 proximate handle 310. Handle 310 includes coupler 312.

The invention is further described by the following numbered paragraphs:

1. A laryngoscope comprising:
a blade comprising:
   an upper blade surface comprising a surface finish comprising a grade value in a range from about 15 to about 30 when measured according to Verein Deutscher Ingenieure (VDI) 3400 Standard; and
   a lower blade surface;
a handle coupled to the blade; and
wherein at least part of the surface finish of the upper blade surface engages soft tissue of a patient during use.
   2. The laryngoscope of paragraph 1 wherein the soft tissue comprises an epiglottis.
   3. The laryngoscope of paragraph 1 wherein the surface finish is configured to non-traumatically engage the soft tissue of the patient.
   4. The laryngoscope of paragraph 1 wherein the grade value of the surface finish on the upper blade surface is in a range from about 18 to about 27 when measured according to Verein Deutscher Ingenieure (VDI) 3400 Standard.
   5. The laryngoscope of paragraph 1 wherein the grade value of the surface finish on the upper blade surface is in a range from about 27 to about 30 when measured according to Verein Deutscher Ingenieure (VDI) 3400 Standard.
   6. The laryngoscope of paragraph 1 wherein the grade value of the surface finish on the upper blade surface is in a range from about 21 to about 24 when measured according to Verein Deutscher Ingenieure (VDI) 3400 Standard.
   7. The laryngoscope of paragraph 1 wherein the lower blade surface further comprises a surface finish having a grade value in a range from about 15 to about 30 when measured according to Verein Deutscher Ingenieure (VDI) 3400 Standard.
   8. The laryngoscope of paragraph 4 wherein the grade value of the surface finish of the lower blade is in a range from about 21 to about 24 when measured according to Verein Deutscher Ingenieure (VDI) 3400 Standard.
   9. The laryngoscope of paragraph 1 further comprising an angled portion on the blade and wherein the surface finish of the upper surface of the blade is positioned at least in part on the angled portion.
   10. A laryngoscope comprising:
a blade comprising:
   a first portion having a first surface average roughness (RA);
   a second portion having a second surface average roughness (RA); and
a handle comprising at least a section that is integrally formed with at least a section of the blade;
wherein at least part of the first portion non-traumatically engages soft tissue of a patient during use.
   11. The laryngoscope of paragraph 10 wherein the first surface average roughness is greater than the second surface average roughness.
   12. The laryngoscope of paragraph 10 wherein the first surface average roughness is in a range from about 0.56 μm to about 3.15 μm.
   13. The laryngoscope of paragraph 10 wherein the first surface average roughness is in a range from about 0.56 μm to about 4.5 μm.
   14. The laryngoscope of paragraph 10 wherein the second surface average roughness is less than about 4.5 μm.
   15. The laryngoscope of paragraph 10 wherein the first surface average roughness is in a range from about 0.8 μm to about 2.24 μm.
   16. The laryngoscope of paragraph 10 wherein the first surface average roughness is in a range from about 1.12 μm to about 1.6 μm.
   17. The laryngoscope of paragraph 10 wherein the second surface average roughness is less than about 3.15 μm.
   18. The laryngoscope of paragraph 10 wherein the second surface average roughness is in a range from about 0.8 μm to about 2.24 μm.
   19. The laryngoscope of paragraph 10 wherein the second surface average roughness is in a range from about 1.12 μm to about 1.6 μm.
   20. The laryngoscope of paragraph 10 wherein the soft tissue engaged by the at least part of the first portion comprises an epiglottis.
   21. A laryngoscope for insertion into an airway of a patient, the device comprising:
a straight blade comprising:
   polymeric material having a surface finish on a portion of the blade comprising a grade value in a range from about 18 to about 27 when measured according to Verein Deutscher Ingenieure (VDI) 3400 Standard; and
a handle comprising polymeric material coupled to the blade; and
wherein the top surface of the blade engages the epiglottis of a patient during use to reduce movement of the epiglottis.
   22. A laryngoscope comprising:
a blade comprising:
   an upper blade surface comprising a surface finish comprising a grade value in a range from about 18 to about 30 when measured according to Verein Deutscher Ingenieure (VDI) 3400 Standard; and
   a lower blade surface;
a handle coupled to the blade; and
wherein at least part of the surface finish of the upper blade surface engages soft tissue of a patient during use.
   23. A laryngoscope comprising:
a blade comprising:
   an upper blade surface comprising a surface finish comprising a grade value in a range from about 18 to about 33 when measured according to Verein Deutscher Ingenieure (VDI) 3400 Standard; and
   a lower blade surface;
a handle coupled to the blade; and
wherein at least part of the surface finish of the upper blade surface engages soft tissue of a patient during use.
   24. A laryngoscope for insertion into an airway of a patient, the device comprising:
a straight blade comprising:
   polymeric material having a surface finish on a portion of the blade comprising a grade value in a range from about 27 to about 30 when measured according to Verein Deutscher Ingenieure (VDI) 3400 Standard; and
a handle comprising polymeric material coupled to the blade; and
wherein the top surface of the blade engages the epiglottis of a patient during use to reduce movement of the epiglottis.
   25. A laryngoscope comprising:
a blade comprising:
   an upper blade surface comprising a surface finish comprising a grade value in a range from about 15 to about 33 when measured according to Verein Deutscher Ingenieure (VDI) 3400 Standard; and
   a lower blade surface;
a handle coupled to the blade; and
wherein at least part of the surface finish of the upper blade surface engages soft tissue of a patient during use.
   26. A laryngoscope comprising:
a blade surfaces having surface finishes in of less than about 33 when measured according to Verein Deutscher Ingenieure (VDI) 3400 Standard; and
a handle coupled to the blade; and
wherein at least part of the surface finishes of the blade engage soft tissue of a patient during use.
   27. The laryngoscope of paragraph 26 wherein the blade further comprises:
a first portion having a first VDI 3400 Standard value in a range from about 24 to about 33; and
a second portion having a second having a first VDI 3400 Standard value in a range from about 15 to about 33.
   28. The laryngoscope of paragraph 26 wherein the blade further comprises:
a first portion having a first surface average roughness (RA): and
a second portion having a second surface average roughness (RA).
   29. The laryngoscope of paragraph 26 wherein the blade further comprises:
a first portion having a first surface average roughness (RA) of less than about 4.5 μm; and
a second portion having a second surface average roughness (RA) of less than about 2.24 μm.
   30. The laryngoscope of paragraph 26 wherein the handle is integrally formed with at least a section of the blade.

Although the present invention and its advantages have been described in detail, it should be understood that various changes, substitutions and alterations can be made herein without departing from the spirit and scope of the invention as defined in the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed:

1. A laryngoscope comprising:
a blade comprising:
an upper blade surface comprising a surface finish comprising a grade value in a range from about 15 to about 30 when measured according to Verein Deutscher Ingenieure (VDI) 3400 Standard; and
a lower blade surface;
a handle coupled to the blade; and
wherein at least part of the surface finish of the upper blade surface engages soft tissue of a patient during use.

2. The laryngoscope of claim 1 wherein the soft tissue comprises an epiglottis.

3. The laryngoscope of claim 1 wherein the surface finish is configured to non-traumatically engage the soft tissue of the patient.

4. The laryngoscope of claim 1 wherein the grade value of the surface finish on the upper blade surface is in a range from about 18 to about 30 when measured according to Verein Deutscher Ingenieure (VDI) 3400 Standard.

5. The laryngoscope of claim 1 wherein the grade value of the surface finish on at least one of the upper blade surface or the lower blade surface is in a range from about 27 to about 30 when measured according to Verein Deutscher Ingenieure (VDI) 3400 Standard.

6. The laryngoscope of claim 1 wherein the lower blade surface further comprises a surface finish having a grade value in a range from about 15 to about 30 when measured according to Verein Deutscher Ingenieure (VDI) 3400 Standard.

7. The laryngoscope of claim 1 wherein at least one of the upper blade or the lower blade surface comprises a surface finish having a grade value in a range from about 21 to about 30 when measured according to Verein Deutscher Ingenieure (VDI) 3400 Standard.

8. The laryngoscope of claim 1 further comprising an angled portion on the blade and wherein the surface finish of the upper surface of the blade is positioned at least in part on the angled portion.

9. A laryngoscope comprising:
a blade comprising:
a first portion having a first surface average roughness (RA);
a second portion having a second surface average roughness (RA);
and
a handle comprising at least a section that is integrally formed with at least a section of the blade;
wherein at least part of the first portion non-traumatically engages soft tissue of a patient during use.

10. The laryngoscope of claim 9 wherein the first surface average roughness is greater than the second surface average roughness.

11. The laryngoscope of claim 9 wherein the first surface average roughness is in a range from about 0.56 μm to about 3.15 μm.

12. The laryngoscope of claim 9 wherein the first surface average roughness is in a range from about 0.56 μm to about 4.5 μm.

13. The laryngoscope of claim 9 wherein the first surface average roughness is in a range from about 0.8 μm to about 3.15 μm.

14. The laryngoscope of claim 9 wherein the first surface average roughness is in a range from about 2.2 μm to about 3.15 μm.

15. The laryngoscope of claim 9 wherein the second surface average roughness is less than about 3.15 μm.

16. The laryngoscope of claim 9 wherein the second surface average roughness is in a range from about 0.8 μm to about 3.15 μm.

17. The laryngoscope of claim 9 wherein the second surface average roughness is in a range from about 2.2 μm to about 3.15 μm.

18. The laryngoscope of claim 9 wherein the soft tissue engaged by the at least part of the first portion comprises an epiglottis.

19. A laryngoscope for insertion into an airway of a patient, the device comprising:
a straight blade comprising:
polymeric material having a surface finish on a portion of the blade comprising a grade value in a range from about 18 to about 30 when measured according to Verein Deutscher Ingenieure (VDI) 3400 Standard;
and
a handle comprising polymeric material coupled to the blade; and
wherein the top surface of the blade engages the epiglottis of a patient during use to reduce movement of the epiglottis.

20. The laryngoscope of claim 19 wherein the portion of the blade comprises:
a first portion of the blade comprising a first surface finish having a first grade value in a range from about 18 to about 30 when measured according to the VDI 3400 standard; and
a second portion of the blade comprising a second surface finish having a second grade value in a range from about 15 to about 30 when measured according to the VDI 3400 standard.
